(19) United States
(12) Patent Application Publication
(10) Pub. No.: US 2018/0228871 A1
(43) Pub. Date: Aug. 16, 2018

(54) METHODS FOR THE MOBILIZATION AND USE OF T-CELLS WITH ENHANCED RECONSTITUTION POTENTIAL AND LIFE-SPAN

(71) Applicant: GlycoMimetics, Inc., Rockville, MD (US)

(72) Inventors: Rachel K. KING, Cabin John, MD (US); John L. MAGNANI, Gaithersburg, MD (US); Ingrid G. WINKLER, Yeerongpilly, Queensland (AU)

(73) Assignee: GlycoMimetics, Inc., Rockville, MD (US)

(21) Appl. No.: 15/750,013

(22) PCT Filed: Aug. 2, 2016
(86) PCT No.: PCT/US2016/045139

§ 371 Date: Feb. 2, 2018

Related U.S. Application Data
(60) Provisional application No. 62/200,628, filed on Aug. 3, 2015.

(51) Int. Cl. A61K 38/19; A61P 37/04; A61K 35/17; A61P 35/00; A61P 31/00; A61K 31/7034; C12N 5/0783

(52) U.S. Cl. CPC A61K 38/193; A61P 37/04; A61K 35/17; C12N 5/0636; A61P 31/00; A61K 31/7034; A61P 35/00

(57) ABSTRACT

The disclosure provides a new source of peripheral blood naive/memory stem cells (i.e. (T_{N})/T_{SCM}/T_{CM}) T-cells with enhanced reconstitution potential and/or longer life spans. It discloses that this particular subset of T-cells can be mobilized by administration of at least one mobilizer in combination with at least one E-Seiectin inhibitor.

New methods for the mobilization and harvesting of T-cell populations with enhanced reconstitution potential and/or enhanced life-span, compositions comprising the same, and methods of using the same in the treatment of many diseases including, not exclusively, infection, autoimmune diseases, and/or cancer are disclosed.

T lymphocytes, or T-cells, are an important component of the body's immune response against a variety of triggers, including infection and cancer. Santoni, F. R. (2015). The immune system as a self-centered network of lymphocytes, Immunol Lett. 2015 August; 166(2):109-16. doi: 10.1016/j.imlet.2015.06.002. Epub 2015 Jun. 16. T-cell adoptive transfer (either autologous or allogeneic) and, possibly, mobilization of endogenous T-cells are two of the multiple strategies for treatment of infection, cancer, and other immune-related disorders. For recent reviews, see: Themeli, M. et al. (2015) New cell sources for T cell engineering and adoptive immunotherapy, Cell Stem Cell. 2015 Apr. 2; 16(4):357-66. doi: 10.1016/j.stem.2015.03.011; Maus M V et al., Adoptive immunotherapy for cancer or viruses. Annu Rev Immunol (2014) 32:189-225.10.1146/annurev-immunol-032713-120136; Butler M O et al. (2011) Establishment of antitumor memory in humans using in vitro-educated CD8+ T cells. Sci Transl Med 3(80):80ra34.10.1126/scitranslmed.3002207; de Aquino et al. (2015), Challenges and future perspectives of T cell immunotherapy in cancer. Immunol Lett. 2015 August; 166(2):117-133. doi: 10.1016/j.imlet.2015.05.018. Epub 2015 Jun. 19.

In adoptive transfer immunotherapy, T-cells can be infused into a patient in their native form or in genetically-modified forms, whereby they express one or more exogenous molecules important for their therapeutic activity. The latter can be, for example, natural T-cell receptors (TCR) or chimeric antigen receptors (CAR). CAR-T cell therapy has been reported to have been successful in the cancer clinic. For review, see, for example, Jena, B. et al. (2014) Driving CAR-Based T-Cell Therapy to Success, Curr Hematol Malig Rep. 2014 March; 9(1): 50-56; June, C. H. Adoptive T cell therapy for cancer in the clinic. J. Clin. Invest. 117, 1466-1476 (2007); Morgan, R. A. et al. Cancer regression in patients after transfer of genetically engineered lymphocytes. Science 314, 126-129 (2006); Pule, M. A. et al. Virus-specific T cells engineered to coexpress tumor-specific receptors: persistence and antitumor activity in individuals with neuroblastoma. Nat. Med. 14, 1264-1270 (2008); and references cited below in CAR-T section.

T-cells are one of the products of hematopoietic stem cell (HSC) differentiation, others including B-cells, neutrophils, and other blood system components. Mobilization of hematopoietic stem cells and their cellular differentiation products can be promoted via the administration of certain growth factors or cytokines. Saraceni et al. (2015) Mobilized peripheral blood grafts include more than hematopoietic stem cells: the immunological perspective, Bone Marrow Transplant. 2015 July; 50(7):886-91. dol: 10.1038/bmt.2014.330. Epub 2015 Feb. 9. For example, granulocyte-colony stimulating factor (G-CSF) has long been used to promote the production of neutrophils in patients with chemotherapy-induced neutropenia and mobilize HSC for transplantation. Deotare et al., 2015. G-CSF-primed bone marrow as a source of stem cells for allografting: revisiting the concept. Bone Marrow Transplant. 2015 Apr. 27. doi: 10.1038/bmt.2015.80. [Epub ahead of print] and Bendall U, et al. (2014), G-CSF From granulopoietic stimulant to bone marrow stem cell mobilizing agent.: Cytokine Growth Factor Rev. 2014 August; 25(4):355-67. doi: 10.1016/j.cytogfr.2014.07.011. Epub 2014 Jul. 23.

It has been reported that human neutrophils and mouse neutrophils express different glycoforms of L-selectin, also known as CD62L. Zöllner O. et al., L-selectin from human, but not from mouse neutrophils binds directly to E-selectin, J Cell Biol. 1997 Feb. 10; 136(3):707-16. In addition, certain populations of both human and mouse T-cells are also CD62L-positive (CD62L). L-selectin contains carbohydrates that bind E-selectin. Graber N. et al., 1990, T cells bind to cytokine-activated endothelial cells via a novel, inducible sialoglycoprotein and endothelial leukocyte adhesion molecule-1, J Immunol. 1990 Aug. 1; 145(3):819-30. E-selectin is also expressed by the bone marrow vascular endothelial cells, where it drives HSC proliferation. Winkler I J et al., 2012, Vascular niche E-selectin regulates hematopoietic stem cell dormancy, self renewal and chemoresistance, Nat Med. 2012 November; 18(11):1651-7. doi: 10.1038/nm.2969. Epub 2012 Oct. 21. Transient administration of a small synthetic E-selectin antagonist (GMI-1070) promotes HSC self-renewal. Id. Subsequently, it has been reported that inhibition of E-selectin with another E-selectin antagonist (GMI-1271) during HSC mobilization with G-CSF results in the mobilization of HSCs with almost 25-fold greater reconstitution potential. Mobilisation of Reconstituting HSC Is Boosted By Synergy Between G-CSF and E-Selectin Antagonist GMI-1271. Conference: 56th Annual Meeting of the American-Society-of-Hematology Location: San Francisco, Calif. Date: Dec. 6-9, 2014. BLOOD Volume: 124 Issue: 21 Published: Dec. 6, 2014.

A possible limitation to the utility of T-cell-mediated therapy is the scarcity of the T-cells themselves, particularly those that the patient's immune system will not reject as foreign, will not cause graft-versus-host-disease, and that will last for a time sufficient to produce an effective treatment. Discussed in, e.g., Nayar S. et al., (2015), Extending the lifespan and efficacies of immune cells used in adoptive transfer for cancer immunotherapies-A review, Oncoimmunology. 2015 Mar. 19; 4(4):e1002720. eCollection 2015. In addition, in cancer patients and those with many other disorders, the TSCM/CM/naïve cell population may be exhausted. This may make it more difficult to collect sufficient number of cells that will persist and function for prolonged periods post autologous transplant as unmodified, TCR-modified, or CAR-T cells. Accordingly, there may be an unmet need for new T-cell sources that meet one or more of these requirements.

### SUMMARY

The disclosure provides a new source of peripheral blood naïve/memory stem cells (i.e. (T_{N}/T_{SCM}//T_{CM}) T-cells with enhanced reconstitution potential and/or longer life spans. It discloses that this particular subset of T-cells can be mobilized by administration of G-CSF in combination with an E-Selectin inhibitor. Other mobilizers can be used (e.g., CXCR4 blockade with VLA-4 blockade, together).

In embodiment 1, the disclosure provides a method of mobilizing to the peripheral blood from a subject T-cells that are either Tₙₐᵢᵥₑ, T_{CM}, T_{SCM}, CD62L^{high}CCR7⁺, CD8⁺CD62L^{high}CCR7⁺, CD8⁺CD62L^{high}, CD44⁻CD8⁺CD62L^{high}, or CD44⁺CD8⁺CD62L^{high}, and combinations thereof, with enhanced reconstitution potential and/or a long life span, the method comprising administering to the subject at least one mobilizer in combination with at least one E-selectin inhibitor.

In embodiment 2, the disclosure provides for a method according to embodiment 1, wherein the at least one mobilizer is G-CSF.

In embodiment 3, the disclosure provides for a method according to any one of embodiments 1 and 2, wherein the at least one E-selectin inhibitor is GMI-1271.

In embodiment 4, the disclosure provides for a method according to any one of embodiments 1, 2, and 3, wherein the G-CSF is administered at a dose from 0.5 μg/kg/day to 50 μg/kg/day.

In embodiment 5, the disclosure provides for a method according to any one of embodiments 1 through 4, wherein the cells are CD62L^{high}CCR7⁺ cells.

In embodiment 6, the disclosure provides for a method of modulating an immune response in a subject in need thereof, wherein the subject suffers from at least one condition selected from cancers, infectious diseases, autoimmune diseases, GVHDs, and transplantations, the method comprising administering the cells according to any one of embodiments 1 through 6.

In embodiment 7, the disclosure provides for the method of embodiment 6. wherein the at least one mobilizer is G-CSF.

In embodiment 8, the disclosure provides for the method according to any one of embodiments 6 and 7, wherein the at least one E-selectin inhibitor is GMI-1271.

In embodiment 9, the disclosure provides for the method according to any one of embodiments 6 through 8, wherein the G-CSF is administered at a dose from 0.5 μg/kg/day to 50 μg/kg/day.

In embodiment 10, the disclosure provides for the method according to any one of embodiments 6 through 9, wherein the cells are CD62L^{high}CCR7⁺ cells.

In embodiment 11, the disclosure provides for a method of producing CAR-T cells with enhanced reconstitution potential and/or a long life span, wherein the CAR-T cells are produced according to any one of embodiments 1 through 5.

In embodiment 12, the disclosure provides for the method of embodiment 11, wherein the at least one mobilizer is G-CSF.

In embodiment 13, the disclosure provides for the method according to any one of embodiments 11 and 12, wherein the at least one E-selectin inhibitor is GMI-1271.

In embodiment 14, the disclosure provides for the embodiment according to any one of embodiments 11-13, wherein the G-CSF is administered at a dose from 0.5 μg/kg/day to 50 μg/kg/day.

In embodiment 15, the disclosure provides for the method according to any one of embodiments 11-14, wherein the cells are CD62L^{high}CCR7⁺ cells.

In embodiment 16, the disclosure provides a method of producing TCR-modified cells with enhanced reconstitution potential and/or a long life span, wherein the TCR-modified cells are produced according to any one of embodiments 1 through 5.

In embodiment 17, the disclosure provides for the method according to embodiment 16, wherein the at least one mobilizer is G-CSF.

In embodiment 18, the disclosure provides for the method according to any one of embodiments 1, wherein the at least one E-selectin inhibitor is GMI-1271.

In embodiment 19, the disclosure provides for the method according to any one of embodiments 11-13, wherein the G-CSF is administered at a dose from 0.5 μg/kg/day to 50 μg/kg/day.

In embodiment 20, the disclosure provides for the method according to any one of embodiments 16-19, wherein the cells are CD62L^{high}CCR7⁺ cells.

In embodiment 21, the disclosure provides for a method of treating cancer, infections, or autoimmune diseases in a subject in need thereof, the method comprising administering to the subject cells are produced according to any one of embodiments 1 through 5 and 11 through 20.

In embodiment 22, the disclosure provides for a method according to embodiment 21 wherein the at least one mobilizer is G-CSF.

In embodiment 23, the disclosure provides for a method according to any one of embodiments 21 and 22, wherein the at least one E-selectin inhibitor is GMI-1271.

In embodiment 24, the disclosure provides for a method according to any one of embodiments 21-23, wherein the G-CSF is administered at a dose from 0.5 μg/kg/day to 50 μg/kg/day.

In embodiment 25, the disclosure provides for a method according to any one of embodiments 21-24, wherein the cells are CD62L^{high}CCR7⁺ cells.

In embodiment 26, the disclosure provides for a method of producing differentiated T-cells, the method comprising culturing cells that are produced according to any one of embodiments 1 through to 5 and 11 through 20.

In embodiment 27, the disclosure provides for a composition comprising a population of T-cells, wherein the T-cells have been produced according to any one of embodiments 1 through 5 and 11 through 20.

In embodiment 28, the disclosure provides for a composition according to embodiment 27, further comprising at least one antibody chosen from antibodies against CD44, CD62L, CD45RO, CCR7, CD45RA, CD62L, CD27, CD28, IL-7Rα, CD95, IL-2Rβ, CXCR3, and LFA-1.

In embodiment 29, the disclosure provides for a composition according to any one of embodiments 27 and 28, further comprising artificial cell growth medium.

### BRIEF DESCRIPTION OF DRAWINGS

Those of ordinary skill in the art will understand that the drawings, described below, are for illustrative purposes only. The drawings are not intended to limit the scope of the present teachings in any way.

The patent or application file contains at least one drawing executed in color. Copies of this patent or patent application publication with color drawings will be provided by the United States Patent and Trademark Office upon request and payment of the necessary fee.

FIG. 1: experimental outline for G-CSF-mediated mobilization of a subset of T-cells in the presence of E-Selectin inhibitor GMI-1271.

FIG. 2: Total leukocytes ×10³ per uL blood in each of the 6 groups (untreated group included as well). Note significant increase with 24 hr administration of GM11271 (˜1.5-fold over G-CSF 3 days alone).

FIG. 3: Total T-cells per uL blood (CD4+ and CD8+ combined). No change with GM11271 co-administration.

FIG. 4: FIG. 4A. CD4+ Tcells per uL blood (no change); FIG. 4B. CD44+CD62hi CD4+ T-cells per uL blood (appear to be decreased by GCSF administration; GMI-1271 co-administration does not appear to boost their numbers in blood).

FIG. 5: FIG. 5A. CD8+ Tcells per uL blood (no changes); FIG. 5B. CD44+CD62hi CD8+ Tcells per uL blood (significantly increased by 24 hrs GMI-1271 co-administration).

FIG. 6: FIG. 6A, CD4+CD62LHi cells in the blood; FIG. 6B: CD8+CD62LHigh in the blood.

FIG. 7: Exemplary cell surface markers and cell populations.

### DESCRIPTION

Unless specifically defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The practice of the present disclosure will employ, unless otherwise indicated, conventional techniques of immunology, tissue culture, molecular biology, chemistry, biochemistry and recombinant DNA technology, which are within the skill of the art. The materials, methods and examples are illustrative only and not limiting. The following is presented by way of illustration and is not intended to limit the scope of the disclosure.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form.

The terms defined below are more fully defined by reference to the specification as a whole.

While the terms used herein are believed to be well understood by one of ordinary skill in the art, the definitions included in this document are set forth to facilitate explanation of the presently-disclosed subject matter.

Following long-standing patent law convention, the terms “a”, “an”, and “the” refer to “one or more” when used in this application, including the claims. Thus, for example, reference to “a cell” includes one cell or a plurality of cells, and so forth.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Throughout this disclosure, various embodiments can be presented in a range format. Numeric ranges are inclusive of the numbers defining the range. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3.8, 4, 5.1, 5.3, and 6. This applies regardless of the breadth of the range.

As used herein the term “comprising” or “comprises” is used in reference to compositions, methods, and respective component(s) thereof, that are open to the inclusion of unspecified elements, whether essential or not.

As used herein the term “consisting essentially of” refers to those elements required for a given embodiment. The term additionally permits the presence of elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

“Hematopoietic stem cells (HSC)” are primitive cells capable of regenerating all blood cells. During development, the site of hematopoiesis translocates from the fetal liver to the bone marrow, which then remains the site of hematopoiesis throughout adulthood. An HSC is a cell with multi-lineage hematopoietic differentiation potential and sustained self-renewal activity. “Self-renewal” refers to the ability of a cell to divide and generate at least one daughter cell with the identical (e.g., self-renewing) characteristics of the parent cell. The second daughter cell may commit to a particular differentiation pathway. For example, a self-renewing hematopoietic stem cell divides and forms one daughter stem cell and another daughter cell committed to differentiation in the myeloid or lymphoid pathway. A committed progenitor cell has typically lost the self-renewal capacity, and upon cell division produces two daughter cells that display a more differentiated (i.e., restricted) phenotype. Hematopoietic stem cells have the ability to regenerate long term multi-lineage hematopoiesis (e.g., “long-term engraftment”) in individuals receiving a bone marrow or cord blood transplant. It is well known in the art that hematopoietic stem cells include pluripotent stem cells, multipotent stem cells (e.g., a lymphoid stem cell), and/or stem cells committed to specific hematopoietic lineages. The stem cells committed to specific hematopoietic lineages may be of T cell lineage, B cell lineage, dendritic cell lineage, Langerhans cell lineage and/or lymphoid tissue-specific macrophage cell lineage. In addition, HSCs also refer to long term HSC (LT-HSC) and short term HSC (ST-HSC). A long term stem cell typically includes the long term (more than three months) contribution to multilineage engraftment after transplantation. A short term stem cell is typically anything that lasts shorter than three months, and/or that is not multilineage. LT-HSC and ST-HSC are differentiated, for example, based on their cell surface marker expression. LT-HSC are CD34-, SCA-1+, Thy1.1+/lo, C-kit+, lin-, CD135-, Slamfl/CD150+, whereas ST-HSC are CD34+, SCA-1+, Thy1.1+/o, C-kit+, lin-, CD135-, Slamfl/CD150+, Mac-1 (CD1lb)lo (“lo” refers to low expression). In addition, ST-HSC are less quiescent (i.e., more active) and more proliferative than LT-HSC. LT-HSC have unlimited self-renewal (i.e., they survive throughout adulthood), whereas ST-HSC have limited self-renewal (i.e., they survive for only a limited period of time).

As used herein, “pharmaceutically acceptable carrier” includes any material, which, when combined with the G-CSF or E-selectin inhibitor or other therapeutic agent, retains its activity and is non-reactive with the subject's immune systems. Examples include, but are not limited to, phosphate buffered saline solutions, water, emulsions such as oil/water emulsions, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well-known conventional methods.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “therapy” refers to “treating” or “treatment” of a disease or condition including inhibiting the disease (slowing or arresting its development), providing relief from the symptoms or side-effects of the disease (including palliative treatment), and relieving the disease (causing regression of the disease). In contrast, prophylactic treatment refers to preventing the disease or condition from occurring in a subject that may be predisposed to the disease but does not yet experience or exhibit symptoms of the disease.

Methods of the presently-disclosed subject matter will now be described.

Mobilization and Harvesting of T-Cells Having Enhanced Reconstitution Potential and/or Longer Life Span in the Presence of G-CSF and an E-Selectin Inhibitor

In one embodiment, human peripheral blood naïve/memory stem cells (i.e. (T_{N}/T_{SCM}/T_{CM}) T-cells with enhanced reconstitution potential and/or longer life spans can be mobilized by administering G-CSF (or another mobilizer) to a subject in the presence of at least one E-Selectin inhibitor. These cells, once harvested, can be used as is or, for example, genetically modified into TCR-modified or CAR-T cells for use in immunotherapy. In one embodiment, the peripheral blood naïve/memory stem cells (i.e. T_{N}/T_{SCM}/T_{CM}) T-cells with enhanced reconstitution potential and/or longer life spans are CD62Lhigh/+CCR7+. In some embodiments, the T-cells of interest are CD8+CD62Lhigh T-cells. In one embodiment, the T-cells of interest can be found within a CD45RO−, CCR7+, CD45RA+, CD62L+, CD27+, CD28+ and IL-7Rα+ T cell compartment characteristic of naïve T-cells, and express large amounts of CD95, IL-2Rβ, CXCR3, and LFA-1. In all embodiments of this disclosure, in this section or elsewhere, the cells of interest have been mobilized by G-CSF (or another mobilizer) in the presence of at least one E-Selectin inhibitor. Other mobilizers can also be used including, without limitation, CXCR4 antagonists (e.g., Plerixafor, Mozobil) with VLA-4 blockade. Their uses are further described below.

G-CSF is a glycoprotein that stimulates the survival, proliferation, differentiation and function of neutrophil granulocyte progenitor cells and mature neutrophils. There are three main biological functions for G-CSF in an living organism, namely: 1. acting on neutrophil precursor cells and myeloid stem cells to drive the differentiation, proliferation, and maturation of neutrophils; 2. activating mature neutrophils to participate in immune response; and 3. synergizing with other hematopoietic growth factors such as stem cell factor, Fit-3 ligand, and GM-CSF to perform hematopoletic functions. At least two forms of recombinant human G-CSF in clinical use are potent stimulants of neutrophil granulopoiesis and have demonstrated efficacy in preventing infectious complications of some neutropenic states. They can be used to accelerate neutrophil recovery from myelosuppressive treatments. The human form of G-CSF was cloned by groups from Japan and the U.S.A. in 1986 (see e.g., Nagata et al. Nature 319: 415-418, 1986). The natural human glycoprotein exists in two forms, one of 175 and the other of 178 amino acids. The more abundant and more active 175 amino acid form has been used in the development of pharmaceutical products by recombinant DNA technology.

Any G-CSF can be used in the methods disclosed herein and thus several embodiments are foreseen. Currently, there are two main categories of rhG-CSF used for therapy available in the market. The first category comprises recombinant proteins expressed by E. coli comprising 175 amino acids with 19 kD in molecular weight and the amino terminus thereof is methionine (Filgrastim); recombinant proteins produced by the mammalian cell CHO comprising 174 amino acids and modified by glycosylation. This category of rhG-CSF is short-acting and typically requires multiple injections daily or weekly for the currently known clinical uses. The second category comprises Filgrastim with pegylation (20 kD-PEG) modification on the N terminal of the protein molecule thereof. The molecular weight of the modified Pegfilgrastim is doubled, which reduces the renal excretion rate, increases the half-life of Filgrastim from 3.5 hours to 15-80 hours and facilitates the clinical use. The rhG-CSF used in both categories is G-CSF monomer, but G-CSF dimers have also been described in the art. Other commercially available recombinant human G-CSF exist, for example, Neupogen and Neulasta, and others are being developed. Bonig et al., 2015. See, for example: Biosimilar granulocyte-colony-stimulating factor for healthy donor stem cell mobilization: need we be afraid? Transfusion. 2015 February; 55(2):430-9. doi: 10.1111/trf.12770. Epub 2014 Jun. 26; Martino M. et al., 2014, Long-active granulocyte colony-stimulating factor for peripheral blood hematopoietic progenitor cell mobilization, Expert Opin Biol Ther. 2014 June; 14(6):757-72. doi: 10.1517/14712598.2014.895809. Epub 2014 Mar. 5; and Hoggatt et al., (2014), New G-CSF agonists for neutropenia therapy, Expert Opin Investig Drugs. 2014 January; 23(1):21-35. doi: 10.1517/13543784.2013.838558. Epub 2013 Sep. 27.

G-CSF can be administered intravenously or by any other suitable method. One of ordinary skill in the art is capable of determining the best route of administration. The G-CSF may be administered to the patient, for example, orally, by subcutaneous injection, by infusion into the blood, or delivered directly to a target tissue site. The G-CSF may be delivered by a single dose, bolus, multiple injections, or by continuous infusion. For example, G-CSF may be injected, infused, or otherwise administered in the blood stream, bone marrow, or any location in the body.

G-CSF can be administered in one or more doses and/or treatment regimens. In one embodiment, G-CSF is administered in an amount ranging from 5 μg/kg to 5 μg/kg/day. In one embodiment, G-CSF is administered at a dose of between 0.5 μg/kg 5 μg/kg/day. One or more treatment cycles may be repeated for a total of three cycles, for example, but any number of cycles is contemplated. The number of treatment cycles per day and the amount per dose may vary during each cycle. For example, depending on the formulation administered, the dose of G-CSF administered may range from about 300 μg Filgastrim (fig) to about 960 μg once a day, or from about 5 μg/kg to about 32 μg/kg once a day. In one embodiment, a proportion of 20 to 50% of the total dose is given as a bolus at the start of treatment and the remaining proportion is administered continuously over the treatment period. The foregoing ranges are exemplary and may vary depending on the size, age, and health of the patient, the route of administration, the number and concentration of other medications the patient is taking, the severity of the patient's condition, the tolerance of the patient to the composition, among other factors. For example, a dose for a 70 kg human may be 480 μg in a 2 ml injection may be an appropriate dose. An optimal G-CSF schedule can be selected by one of ordinary skill in the art according to the objectives of this disclosure.

Other non-G-CSF “mobilizers” that have been, and/or can be, utilized for mobilization of HSC and could be used to mobilize the T-cells disclosed herein include CXCR4 antagonists, combination of VLA-4 inhibitor with ADM3100 (a CXCR4 inhibitor), or others. In one embodiment, the mobilizer can be chosen from SCF, Flt3 Ligand, IL-3, IL-6 and IL-11, which renew primitive, pluripotent progenitor cells that are capable of sustaining hematopoiesis. In another embodiment, cyclophosphamide can be combined with G-CSF.

According to this disclosure, one or more E-Selectin inhibitors are administered in combination with G-CSF. The combined use of both agents suitably overlap so that the therapeutic effect of one agent (i.e. the time period post use where a measurable benefit to the patient is observed) is concurrent, at least at some point, with the period of therapeutic effect of the second agent. The two types of agents work together to achieve the desired effect of T-cells having enhanced reconstitution potential and/or long effective life spans. These two types of agents may be administered together or sequentially. As used herein, “together” is used to mean that the two types agents are administered concurrently. They can be administered in the same composition or in separate compositions. In contrast to “together,” “sequentially” is used herein to mean that the gap between administering one agent and the other is significant i.e. the first administered agent may no longer be present in the bloodstream in a therapeutic amount when the second agent is administered. Either may be administered first or later.

E-selectin (CD62E) is a cell adhesion molecule that is expressed on activated endothelial cells and plays an important role in leukocyte recruitment to the site of vascular injury. GMI-1271 is designed to mimic the bioactive conformation of the sialyl-Lex carbohydrate binding domain of E-selectin and is a specific E-selectin inhibitor. Myers et al., 2014, E-Selectin Inhibitor GMI-1271 Works in Combination with Low-Molecular Weight Heparin to Decrease Venous Thrombosis and Bleeding Risk in a Mouse Model, Blood: 124 (21), 593-593. In one embodiment, the at least one E-Selectin inhibitor is the compound GMI-1271 or Sodium (1R, 3R, 4R, 5S)-3-({2-N-acetylamino-2-deoxy-3-O-[(1 S)-1-carboxylato-2-cyclohexylethyl]-β-D-galactopyranosyl)}oxy)-4-({6-deoxy-α-L-galactopyranosyl)}xy)-5-ethyl-cyclohexan-1-yl-(38-oxo-2,5,8,11,14,17,20,23,26,29,32,35-dodecaoxa-39-azahentetracontan-41-yl) carboxamide represented by the following structure:

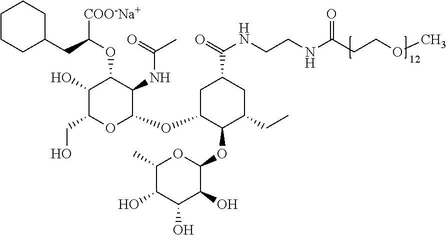

In other embodiments, the at least one E-Selectin inhibitor is chosen from GMI-1271, GMI-1070, GMI-1359 and other glycomimetics.

Any other E-Selectin inhibitor(s) can be used in the methods disclosed herein. In some embodiments, the at least one E-Selectin inhibitor is chosen from sialyl Lewis^{x} (sLe^{x}) or sLe^{x} mimetics. E-Selectin inhibitors can also be chosen from other small molecule glycomimetic antagonists of E-Selectin, antibodies directed to E-Selectin, aptamers to E-Selectin, peptides and peptidomimetics directed to E-Selectin.

As with G-CSF, one or more E-Selectin inhibitors can be administered intravenously or by any other suitable method. One of ordinary skill in the art is capable of determining the best route of administration. The at least one E-Selectin inhibitor may be administered to the patient, for example, orally, by subcutaneous injection, by infusion into the blood, or delivered directly to a target tissue site. The at least one E-Selectin inhibitor(s) may be delivered by a single dose, bolus, multiple injections, or by continuous infusion. For example, the at least one E-Selectin inhibitor may be injected, infused, or otherwise administered in the blood stream, bone marrow, or any location in the body.

The at least one E-Selectin inhibitor can be administered in one or more doses and treatment regimens, which may be the same or different. In one embodiment, the at least one E-Selectin inhibitor is administered in an amount ranging from about 1 mg/kg to about 50 mg/kg once a day. In another embodiment, the at least one E-Selectin inhibitor is administered in an amount ranging from about 1 mg/kg to about 5 mg/kg once a day. In other embodiments, the dosage may be at any dosage including, but not limited to, about 1 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg. In other embodiments, is administered in any of these amounts and ranges more than once a day, every other day, every two days, etc. One of more treatment cycles may be repeated for a total of three cycles, but any number of cycles is contemplated. The number of treatments per day and the amount per dose may vary during each cycle.

In one embodiment, the at least one E-Selectin inhibitor is GMI-1271. In one embodiment, GMI-1271 is administered in an amount of 0.5 mg/kg to 50 mg/kg on the second and third day, with administration of 10 μg/Kg/day of G-CSF on days 0 to 3. In one embodiment, the T-cells of the disclosure are mobilized after 4-6 days of GCSF administration. This mobilization can be boosted when GMI-1271 is co-administered for 4 days to 12 hours before blood harvest.

T-cell mobilization in response to G-CSF in the presence or absence of an E-Selectin inhibitor can be assayed by any known method. Blood and plasma samples can be sampled at baseline and at different stages of treatment with these agents.

Prior to expansion and genetic modification of the peripheral blood naïve/memory stem cells (i.e. T_{N}/T_{SCM}/T_{CM}) T-cells with enhanced reconstitution potential and/or longer life spans disclosed herein as described below for the production of CAR-T cells, a source of the T-cells disclosed herein is obtained from a subject. The T-cells disclosed herein, obtained after mobilization with, for example, G-CSF in the presence of at least one E-Selectin inhibitor, can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, T-cells can be obtained from a unit of blood collected from a subject using any number of techniques known to the skilled artisan, such as Ficoll separation. A person of ordinary skill in the art would recognize that multiple rounds of selection can also be used in the context of this disclosure.

In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the disclosure, the cells are washed with phosphate buffered saline (PBS). In an alternative embodiment, the wash solution lacks calcium and may lack magnesium or may lack many if not all divalent cations In another embodiment, blood mononuclear cells (BMNC) can be isolated by any method such as density gradient centrifugation. The T-cells can be separated from BMNC and then further separated into different T-cell populations by positive and negative selection using well-known methods such as, for example, magnetic beads, or isolated by affinity to a solid phase having a specific antibody or by FACS of labeled cells. After harvesting, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In another embodiment, T-cells are isolated from peripheral blood lymphocytes by lysing the red blood cells and depleting the monocytes, for example, by centrifugation through a PERCOLLgradient or by counterflow centrifugal elutriation. A specific subpopulation of T-cells, such as CD62L+, CD3⁺, CD28⁺, CD⁴⁺, CD8+, CD45RA+, and CD45RO+ T-cells, can be further isolated by positive or negative selection techniques. For example, in one embodiment, T-cells are isolated by incubation with anti-CD3/anti-CD28 (i.e., 3×28)-conjugated beads, such as DYNABEADS M-450 CD3/CD28 T, for a time period sufficient for positive selection of the desired T cells. In another embodiment, T-cells are isolated by incubation with anti-CD62L beads and anti-CD8 beads. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours.

Enrichment of a T-cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD8⁺ cells by negative selection, a monoclonal antibody cocktail typically includes antibodies to CD14, CD20, CD11b, CD16, HLA-DR, and CD4. In certain embodiments, it may be desirable to enrich for or positively select for regulatory T-cells which typically express CD8+, CD25+, CD62L^{hi}, GITR+, and FoxP3+. Alternatively, in certain embodiments, T regulatory cells are depleted by anti-C25 conjugated beads or other similar method of selection.

By practicing these methods, one or more T-cell populations of the disclosure can be isolated. A “cell population,” as that term is used herein, encompasses a population of cells in which the majority of cells is of a same cell type or has a same characteristic. One convenient way to class single cells as part of a cell population is to determine the level of a cell surface marker of a given cell population on the single cell. The term “cell surface marker” and “extracellular cell marker” are used interchangeably herein. For example, T-cells can be identified and classed based on the presence or absence, or relative abundance, of the CD62L, CCR7, CD4+ or CD8+ markers; thus one cell population can be CD4+ T cells, or T helper cells. Such markers and classifications are well-known in the art and any suitable method of classification may be used (See, e.g., detailed methods described in Appay, V. et al., Phenotype and function of human T lymphocyte subsets: consensus and issues, Cytometry A. 2008 November; 73(11):975-83. doi: 10.1002/cyto.a.20643. Review and/or De Rosa et al., 11-color, 13-parameter flow cytometry: identification of human naive T cells by phenotype, function, and T-cell receptor diversity, Nat Med. 2001 February; 7(2):245-8; US Patent Publication No. 2015/0118247, Gattinoni, L. et al. 2013, Moving T memory stem cells to the clinic, Blood 121(4): 567-568; Gattinoni L, et al. Wnt signaling arrests effector T cell differentiation and generates CD8+ memory stem cells. Nat Med. 2009 July; 15(7):808-13. doi: 10.1038/nm.1982. Epub 2009 Jun. 14 and single figure in Restifo Big bang theory of stem-like T cells confirmed Blood. 2014 July 24; 124(4):476-7. doi: 10.1182/blood-2014-06-578989..

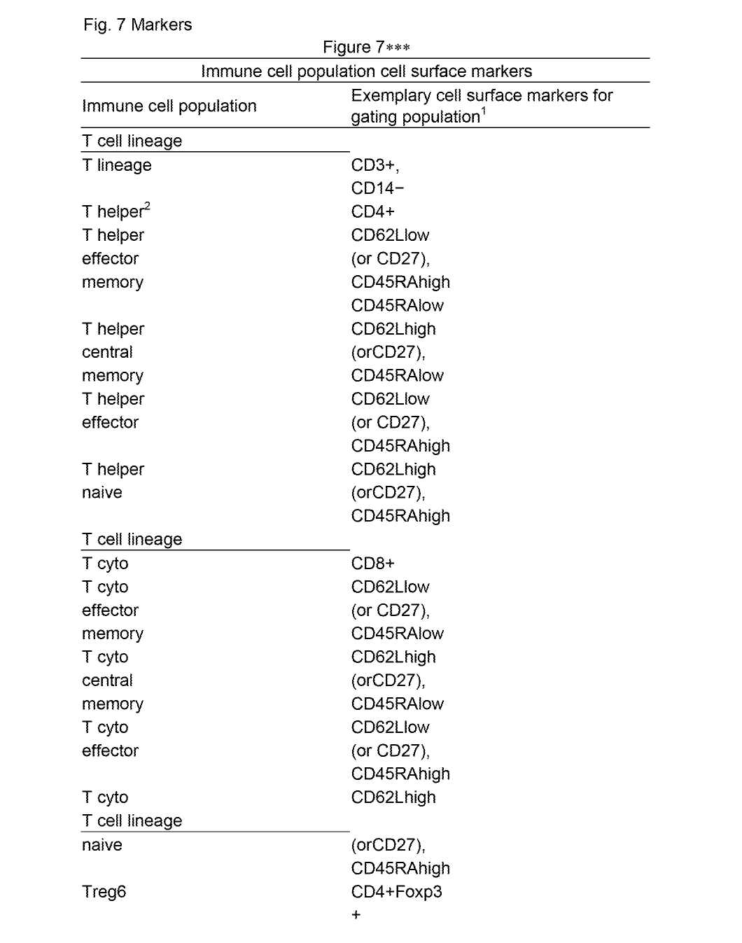

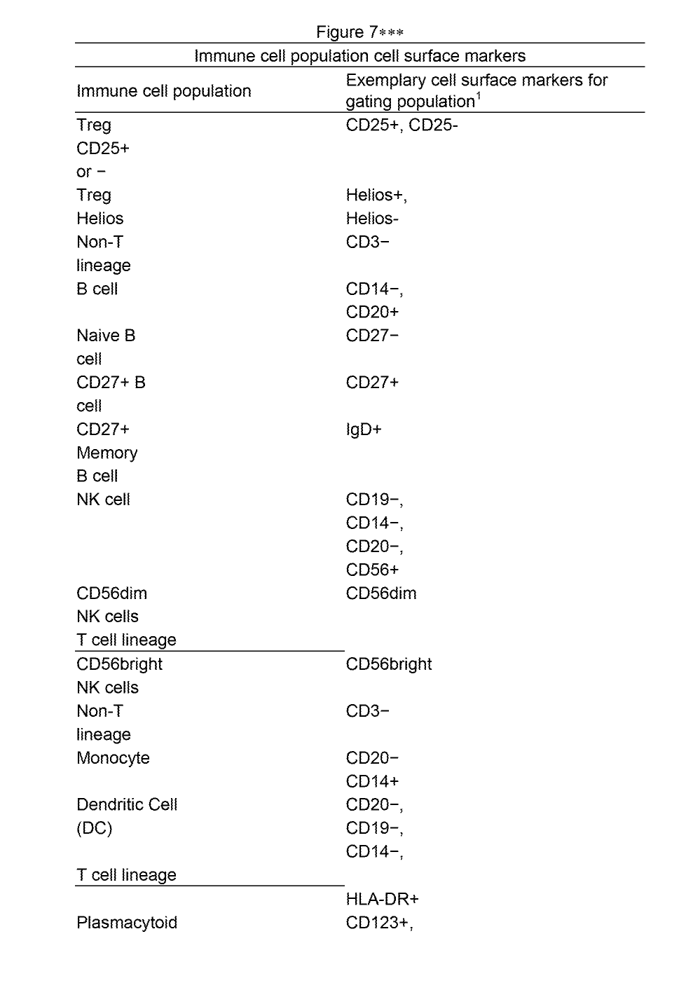

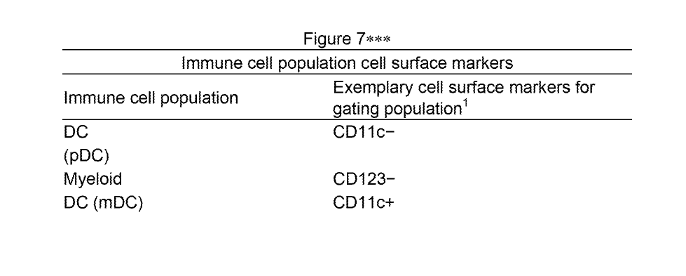

Some additional exemplary cell surface markers and cell populations are shown in FIG. 7. A cell population can also be a subpopulation of another cell population. For example, the T helper cell population is a subpopulation of the T cell lineage population, and the T helper effector population is a subpopulation of the T helper population. Other examples of cell populations that are subpopulations of another cell population are shown in FIG. 7.

After the T-cells have been isolated and sorted into different subpopulations, the population of interest for the methods of this disclosure can be quantified and, if desired, harvested. In one embodiment of these harvested cells, the peripheral blood naïve/memory stem cells (i.e. T_{N}/T_{SCM}/T_{CM}) T-cells with enhanced reconstitution potential and/or longer life spans are CD62L^{high/+}CCR7⁺. In some embodiments, the T-cells of interest are CD8⁺CD62L^{high} T-cells. In one embodiment, the T-cells of interest can be found within a CD45RO⁺, CCR7⁺, CD45RA⁺, CD62L⁺, CD27⁺, CD28⁺ and IL-7Rα⁺ T cell compartment characteristic of naive T cells, and express large amounts of CD95, IL-2Rβ, CXCR3, and LFA-1. In some of embodiments, the T-cells of interest have been selected on the basis of markers and methods described in Appay, V. et al., Phenotype and function of human T lymphocyte subsets: consensus and issues, Cytometry A. 2008 November; 73(11):975-83. doi: 10.1002/cyto.a.20643. Review and/or De Rosa et al., 11-color, 13-parameter flow cytometry: identification of human naive T cells by phenotype, function, and T-cell receptor diversity, Nat Med. 2001 February; 7(2):245-8.

Mobilization of the T-cells of interest is considered to have happened if administering the combination treatment disclosed herein leads to an increase in the population of desired cells in the peripheral blood. Mobilization can occur in about 1 hr, 2 hrs, 3 hrs, 4 hrs, 6 hrs, 8 hrs, 10 hrs, 12 hrs, 14 hrs, 16 hrs, 18 hrs, 20 hrs, 22 hrs, 24 hrs, 26 hrs, 28 hrs, or 30 hours after combination treatment and accumulation of the T-cells of interest including CD8⁺CD62L^{high} T-cells in the blood may peak in about 1 hr, 2 hrs, 3 hrs, 4 hrs, 6 hrs, 8 hrs, 10 hrs, 12 hrs, 14 hrs, 16 hrs, 18 hrs, 20 hrs, 22 hrs, 24 hrs, 26 hrs, 28 hrs, 30 hrs, 40 hrs, 50 hrs, 60 hrs, 65 hrs, 66 hrs, 67 hrs, 68 hrs, 69 hrs, 70 hrs, 71 hrs, 72 hrs, 73 hrs, 74 hrs, 75 hrs, 76 hrs, 77 hrs, 78 hrs, 79 hrs, 80 hrs, 81 hrs, 82 hrs, 83 hrs, 84 hrs, 85 hrs, 86 hrs, 87 hrs, 88 hrs, 89 hrs, 90 hrs, 91 hrs, 92 hrs, 93 hrs, 94 hrs, 95 hrs, 96 hrs, 97 hrs, 98 hrs, 99 hrs, 100 hrs, 101 hrs, 102 hrs, 103 hrs, 104 hrs, 105 hrs, 106 hrs, 107 hrs, 108 hrs, 109 hrs, and/or 110 hrs after administration.

The cells of interest are mobilized by G-CSF or another mobilizer in the presence of at least one E-Selectin inhibitor. In one embodiment, the peripheral blood naïve/memory stem cells (i.e. T_{N}/T_{SCM}/T_{CM}) T-cells with enhanced reconstitution potential and/or longer life spans are CD62L^{high/+}CCR7⁺. In some embodiments, the T-cells of interest are CD8⁺CD62L^{high} T-cells. In one embodiment, the T-cells of interest can be found within a CD45RO⁻, CCR7⁺, CD45RA⁺, CD62L⁺, CD27⁺, CD28⁺ and IL-7Rα⁺ T-cell compartment characteristic of naive T-cells, and express large amounts of CD95, IL-2Rβ, CXCR3, and LFA-1. In one embodiment, at least the latter are reported in the literature as having enhanced reconstitution potential and/or long effective life spans. Gattinoni, L. et al. A human memory T cell subset with stem cell-like properties, Nat Med. 2011 Sep. 18; 17(10):1290-7; Stemberger et al. (2014), Lowest numbers of primary CD8(+) T cells can reconstitute protective immunity upon adoptive immunotherapy, Blood. 2014 July 24; 124(4):628-37. doi: 10.1182/blood-2013-12-547349. Epub 2014 May 22 and Graef et al. (2014) Serial transfer of single-cell-derived immunocompetence reveals stemness of CD8(+) central memory T cells, Immunity. 2014 July 17; 41(1):116-26. doi: 10.1016/j.immuni.2014.05.018. If the T-cells have enhanced reconstitution potential and/or long effective life span by at least one method, the cells are within the scope of this disclosure. The T-cells' enhanced reconstitution potential can be assayed by any of a variety of methods known to one of ordinary skill in the art. In one embodiment, the T-cells' enhanced reconstitution potential is assayed by evaluating their capacity to self-renew with homeostatic signals as well as their multipotency after T-cell receptor activation. In one embodiment, this assay is done as described in Gattitoni, 2011, supra. In one embodiment, the reconstitution potential is measured after transplantation to mice, by methods known in the art.

The T-cells' effective life span can be assayed by any of a variety of methods known to one of ordinary skill in the art. In one embodiment, the T-cells' enhanced life span is assayed by evaluating their long-term replicative and survival capacities, compared with T_{CM} and T_{EM} cells. In one embodiment, this assay is done as described in Gattitoni, 2011, supra. In another embodiment, the T-cells' enhanced life span is assayed, after they have been converted into CAR-T cells, by assaying their anti-tumor efficacy as adoptively transferred CAR-T cells. In one embodiment, this assay is done as described in Gattitoni, 2011, supra. In one embodiment, the cells have enhanced life span if they are capable of long term persistence at more than 0.1% PMBC in vivo for at least 10 years. In one embodiment (e.g., in mice) this will be 12 weeks post-transplant (=20% lifespan).

Methods of maintaining or culturing T-cells are well known in the art. In one embodiment, the cells of interest are cultured as described in Gattitoni, 2011, supra.

Modulation of Immune Response with T-Cells Having Enhanced Reconstitution Potential and/or a Long Effective Life Span Obtained by their Mobilization into the Blood in the Presence of G-CSF and an E-Selectin Inhibitor

A subject's immune response to a variety of stimuli or disorders can be modulated by T-cells having enhanced reconstitution potential and/or long effective life spans that are mobilized by administering G-CSF to the subject in the presence of at least one E-Selectin inhibitor. In another embodiment, the subject's immune response to a variety of stimuli or disorders can be modulated by T-cells having enhanced reconstitution potential and/or long effective life spans that are mobilized by administering another T-cell mobilizer to the subject (instead of or together with G-CSF) in the presence of at least one E-Selectin inhibitor. In some embodiments, the T-cell mobilizer can be chosen CXCR4 blockade, with VLA-4 blockade. In one embodiment, cell mobilization refers to the increase in the number of desired T-cells in the peripheral blood.

In one embodiment of the T-cells that can be mobilized by the methods disclosed herein (T-cells of interest), the peripheral blood naïve/memory stem cells (i.e. (T_{N})/T_{SCM}) T-cells with enhanced reconstitution potential and/or longer life spans, are CD62L^{high/+}CCR7⁺. In some embodiments, the T-cells of interest are CD8⁺CD62L^{high} T-cells. In one embodiment, the T-cells of interest can be found within a CD45RO⁻, CCR7⁺, CD45RA⁺, CD62L⁺, CD27⁺, CD28⁺ and IL-7Rα⁺ T cell compartment characteristic of naive T cells, and express large amounts of CD95, IL-2Rβ, CXCR3, and LFA-1. In one embodiment, the T-cells and/or mobilization methods disclosed herein can be used in the treatment of at least one condition chosen from cancers, inflammations, infections, autoimmune disorders, preventing graft rejection, and/or transplantations, in a subject in need thereof.

The at least one cancer to be treated may be chosen from cancers whose treatment benefits from the increase in T-cells having enhanced reconstitution potential and/or a long effective life span, including brain, breast, pancreatic, liver, kidney, lung, spleen, gall bladder, anal, testicular, ovarian, cervical, skin, bone, blood, and/or colon cancer. In these situations, the benefit may come from the ability of the disclosed T-cells to have been further modified to be specifically adapted to target cancer cells for destruction.

The infections may be chosen from viral infections, bacterial infections, and other known infections. In these situations, the benefit may come from the ability of the disclosed T-cells to have been further modified to be specifically adapted to target infected cells for destruction, relying, for example, on the use of receptors that bind viral antigens.

The term “autoimmune disease” as used herein is defined as a disorder that results from an autoimmune response. An autoimmune disease is the result of an inappropriate and excessive response to a self-antigen. In one embodiment, examples of autoimmune diseases include but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, and ulcerative colitis, among others. In one embodiment, the disclosure provides for treating any autoimmune disease in which chemotherapy and/or immunotherapy in a patient results in significant immunosuppression in the patient in these situations, the benefit comes from the ability of the T-cells to reconstitute immunity.

In one embodiment, the method comprises administering to the subject an effective amount of T-cells with enhanced reconstitution potential and/or a long effective life span, wherein the T-cells have been obtained by mobilization with G-CSF and at least one E-selectin inhibitor.

An “effective amount” as used herein means an amount which provides at least one benefit chosen from therapeutic and prophylactic benefits.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated. For example, in the case of cancer, a therapeutic effect can be killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

In another embodiment, the treatment consists of in vivo mobilization (without isolation followed by administration) of the T-cells with enhanced reconstitution potential and/or a long effective life span, wherein the T-cells have been obtained by mobilization with G-CSF and an E-selectin inhibitor. The method may be administered to a patient before undergoing treatment of cancer, infections, autoimmune disorders, graft versus host disease, or transplantation, or to a donor, from whom such cells may be transplanted to a recipient later.

In some embodiments, the term “treatment” means the slowing down, interruption, arrest, reversal or stoppage of the progression of the disease, which does not necessarily require the complete elimination of all the signs and symptoms of the disease. Furthermore, it is not necessary for the treatment to show effectiveness in 100% of the patients treated, rather, the term “treatment” is intended to mean that a statistically significant proportion of patients can be treated effectively, in such a way that the symptoms and clinical signs show at least an improvement. The person skilled in the art can easily establish whether the proportion is statistically significant using various statistical methods (e.g. confidence intervals, determination of them p value, Student's t-test, Mann-Whitney test etc.). Confidence intervals have a confidence of at least 90%, at least 95%, at least 97%, at least 98% or at least 99%. The p values are 0.1, 0.05, 0.01, 0.005 or 0.0001.

In one embodiment, the T-cells are mobilized as previously disclosed. Once mobilized, the T-cells can be collected or harvested and then transplanted back into the same subject (autologous transplantation) or into another subject (allogenic transplantation) in need thereof. As used herein, the term “autologous” refers to any material derived from the same individual to which it is later to be re-introduced into the individual. “Allogeneic” refers to a graft derived from a different animal (e.g., human) of the same species.

In another embodiment, the treatment consists of in vivo mobilization (without isolation followed by administration) of the T-cells with enhanced reconstitution potential and/or a long effective life span, wherein the T-cells have been obtained by mobilization with G-CSF and an E-selectin inhibitor.

Production of CAR-T Cells with T-Cells Having Enhanced Reconstitution Potential and/or a Long Effective Life Span Obtained by Mobilization in the Presence of G-CSF and an E-Selectin Inhibitor

In one embodiment, T-cells having enhanced reconstitution potential and/or long effective life spans that have been mobilized by administering, for example, G-CSF to a subject in the presence of at least one E-Selectin inhibitor can be used as is in immunotherapy or adoptive T-cell transfer/transplantation. In one embodiment, the peripheral blood naïve/memory stem cells (i.e. T_{N}/T_{SCM}/T_{CM}) T-cells with enhanced reconstitution potential and/or longer life spans are CD62L^{high}CCR7⁺. In some embodiments, the T-cells of interest are CD8⁺CD62L^{high} T-cells. In some embodiments, the T-cells of interest are CD8⁺CD62L^{high}CCR7⁺ T-cells. In one embodiment, the T-cells of interest can be found within a CD45RO⁻, CCR7⁺, CD45RA⁺, CD62L⁺, CD27⁺, CD28⁺ and IL-7Rα⁺ T cell compartment characteristic of naive T cells, and express large amounts of CD95, IL-2Rβ, CXCR3, and LFA-1.

In addition, in another embodiment, these same T-cells having enhanced reconstitution potential and/or long effective life spans that have been mobilized by administering, for example, G-CSF to a subject in the presence of an E-Selectin inhibitor can be used for the production of CAR-T cells. While CARs can trigger T-cell activation in a manner similar to an endogenous T-cell receptor, a major impediment to the clinical application of this technology to date has been limited in vivo expansion of CAR-T cells and rapid disappearance of the cells after infusion. Thus, the T-cells disclosed herein are expected to be beneficial, persistent, and/or provide for long-term effective treatments of cancer, infections, inflammation, and/or autoimmune disease.

After collection, the T-cells disclosed in the previous paragraphs, for example CD62L^{high/+}CCR7⁺, are genetically engineered to produce special receptors on their surface called chimeric antigen receptors (CARs). CARs are proteins that allow the T-cells to recognize a specific protein (antigen) on, for example, tumor cells. These engineered CAR-T cells are then grown in the laboratory until they number in the billions. The expanded population of CAR-T cells is then infused into the patient. In the treatment of cancer, after the infusion, the T-cells multiply in the patient's body and, with guidance from their engineered receptor, recognize and kill cancer cells that harbor the antigen on their surfaces. In another embodiment, one can engraft CARs in naturally occurring regulatory T cells (nTregs) to produce “loss of function” of an unwanted T-cell response that causes inflammation, thereby ameliorating an ongoing autoimmune disorder. See, e.g., Dotti, G. (2014) The Other Face of Chimeric Antigen Receptors, Molecular Therapy (2014); 22 5, 899-900. doi:10.1038/mt.2014.58.

Genetic engineering of CAR-T cells is well-described in the art and can be performed by any molecular biology technique routinely available to one of ordinary skill in the art. See, e.g., US Patent Publication No. 20150118202. In one embodiment, the CAR disclosed herein comprises a target-specific binding element otherwise referred to as an antigen binding moiety. See, e.g., U.S. Pat. No. 8,975,071. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen moiety domain in the CAR disclosed herein include those associated with viral, bacterial and parasitic infections, autoimmune disease and/or cancer cells.

In one embodiment, CAR-T cells can be used for treating a variety of cancers after transplantation into a cancer patient. They have shown promise in the treatment of various cancers, including advanced acute lymphoblastic leukemia (ALL) and lymphoma.

CAR-T cell manufacturing and delivery may include the following steps: (1) leukapheresis: apheresis in which a patient's T cells are harvested from peripheral blood; (2) T-cell activation: T cells are activated using Ab-coated beads that serve as artificial dendritic cells (DCs); (3) transduction or transfection: T cells are genetically transduced or transfected ex vivo with a construct encoding the anti-gene target chimeric antigen receptor; (4) expansion: gene-modified cells undergo further ex vivo expansion; (5) chemotherapy: the patient receives a preparative lymphodepleting regimen before T-cell infusion; (6) infusion: genetically engineered T cells are infused into the patient Levine, B. L. Performance-enhancing drugs: design and production of redirected chimeric antigen receptor (CAR) T cells, Cancer Gene Therapy (2015) 22, 79-84; doi:10.1038/cgt.2015.5; published online 13 Feb. 2015. This disclosure improves on the quality of the cells that are harvested in step (1).

Methods for harvesting T-cells for CAR-T cell production and using CAR-T cells are well-understood by one of ordinary skill in the art and have been used for described and summarized in a variety of journal articles. They can be modified to lead to the selection and harvest of the specific T-cell subsets of this disclosure. See, for example, Themeli et al., (2015); Sharpe et al. (2015) Genetically modified T cells in cancer therapy: opportunities and challenges, Disease Models and Mechanisms, 8(4) 337-350; Riddell et al., 2013. Chimeric Antigen Receptor Modified T Cells—Clinical Translation in Stem Cell Transplantation and Beyond, Biol Blood Marrow Transplant. 2013 January; 19(10): S2-S5; Kershaw et al. (2014) Clinical application of genetically modified T cells in cancer therapy, Clinical & Translational Immunology (2014) 3, e16; doi:10.1038/cti.2014.7; Brentjens R J, Davila M L, Riviere I, et al. CD19-targeted T cells rapidly induce molecular remissions in adults with chemotherapy-refractory acute lymphoblastic leukemia. Sci Transl Med. 2013; 5:177ra38; Grupp S A, Kalos M, Barrett D, et al. Chimeric antigen receptor-modified T cells for acute lymphoid leukemia. N Engl J Med. 2013; 368:1509-1518; Lee D W, Shah N N, Stetler-Stevenson M, et al. Anti-CD19 chimeric antigen receptor (CAR) T cells produce complete responses with acceptable toxicity but without chronic B-cell aplasia in children with relapsed or refractory acute lymphoblastic leukemia (ALL) even after allogeneic hematopoietic stem cell transplantation (HSCT) [abstract]. Blood. 2013; 122:abstr 68; Davila M L, Riviere I, Wang X. et al. Efficacy and toxicity management of 19-28z CAR T cell therapy in B cell acute lymphoblastic leukemia. Sci Transl Med. 2014; 6:224ra25; Park J H, Riviere I, Wang X, et al. CD19-targeted 19-28z CAR modified autologous T cells induce high rates of complete remission and durable responses in adult patients with relapsed, refractory B-cell ALL [abstract]. Blood. 2014; 124:abstr 382; and Levine, B. L. Performance-enhancing drugs: design and production of redirected chimeric antigen receptor (CAR) T cells, Cancer Gene Therapy (2015) 22, 79-84; doi:10.1038/cgt.2015.5; published online 13 Feb. 2015.

In one embodiment, CAR-T cells are expanded, washed and concentrated with phosphate buffered saline and formulated into 100 ml of sterile normal saline supplemented with 5% Albumex20. The CAR-T cells can be administered in an appropriate cell dose, as determined by one of ordinary skill in the art. In one embodiment, the CAR-T cells are administered at a minimum of 1×10⁸ viable cells. The maximum number of reinfused or transplanted T-cells is typically determined during clinical trials done by one of ordinary skill in the art.

Any chimeric antigen receptors (CARs) can be used with the cells disclosed herein. In one embodiment, CARs are chimeric constructs composed of several domains derived from different proteins, namely: (1) an antigen recognition domain that is usually taken from an Ab, (2) a CD34 T-cell co-receptor signaling domain, and (3) a costimulatory domain required for T-cell activation during antigen presentation. Levine, 2015. Any tumor antigen, T-specific antigen/Tumor associated antigen can be targeted. Tumor antigens are proteins that are produced by tumor cells that elicit an immune response, particularly T-cell mediated immune responses. See, e.g., Vigneron N., (2015), Human Tumor Antigens and Cancer Immunotherapy, Biomed Res Int. 2015; 2015:948501. doi: 10.1155/2015/948501. Epub 2015 Jun. 16. The selection of the antigen binding moiety disclosed herein will depend on the particular type of cancer to be treated. Tumor antigens are well known in the art and include, for example, a glioma-associated antigen, carcinoembryonic antigen (CEA), .beta.-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. The type of tumor antigen referred to in the disclosure may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alphafetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In one embodiment, the antigen binding domain in the CAR binds to CD19. In one embodiment, the tumor antigen is associated with a hematologic malignancy. In another embodiment, the tumor antigen is associated with a solid tumor. In yet another embodiment, the tumor antigen is selected from the group consisting, CD20, CD22, ROR1, mesothelin, CD33/IL3Ra, c-Met, PSMA, Glycolipid F77, EGFRvIII, GD-2, NY-ESO-1 TCR, MAGE A3 TCR, and any combination thereof. In one embodiment, the tumor antigen comprises one or more antigenic cancer epitopes associated with a malignant tumor. Malignant tumors express a number of proteins that can serve as target antigens for an immune attack. In one embodiment, these molecules include but are not limited to tissue-specific antigens such as MART-I, tyrosinase and GP 100 in melanoma and prostatic acid phosphatase (PAP) and prostate-specific antigen (PSA) in prostate cancer. In other embodiments, other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other embodiments for target antigens in B-cell lymphoma. Some of these antigens (CEA, HER-2, CD19, CD20, idiotype) have been used as targets for passive immunotherapy with monoclonal antibodies with success.

In one embodiment, the costimulatory signaling region in the CAR comprises the intracellular domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof. In one embodiment, the antigen binding domain in the CAR is an antibody or an antigen-binding fragment thereof. In one embodiment, the antigen-binding fragment is a Fab or a scFv.

In one embodiment, the CAR-T cells persist in the human for at least three months after administration. In another embodiment, the persisting population of CAR-T cells persists in the human for at least four months, five months, six months, seven months, eight months, nine months, ten months, eleven months, twelve months, two years, or three years after administration. In one embodiment, the CAR-T cells disclosed herein are able to replicate in vivo resulting in long-term persistence in the blood or bone marrow that can lead to sustained anti-tumor effects. The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. In one embodiment, the CAR-T cells progeny can comprise memory T-cells.

Production of T-Cells Carrying Genetically Modified T-Cell Receptors (TCR-Cells) with T-Cells Having Enhanced Reconstitution Potential and/or a Long Effective Life Span Obtained by Mobilization in the Presence of G-CSF and an E-Selectin Inhibitor

In one embodiment, T-cells having enhanced reconstitution potential and/or long effective life spans that have been mobilized by administering, for example, G-CSF to a subject in the presence of at least one E-Selectin inhibitor can be used as is in immunotherapy or adoptive T-cell transfer/transplantation after having been modified to express certain T-cell receptors. In one embodiment, the peripheral blood naïve/memory stem cells (i.e. (T_{N}/T_{SCM}/T_{CM}) T-cells with enhanced reconstitution potential and/or longer life spans are CD62L^{high/+}CCR7⁺. In some embodiments, the T-cells of interest are CD8⁺CD62L^{high} T-cells. In some embodiments, the T-cells of interest are CD8⁺CD62L^{high}CCR7⁺ T-cells. In one embodiment, the T-cells of interest can be found within a CD45RO⁻, CCR7⁺, CD45RA⁺, CD62L⁺, CD27⁺, CD28⁺ and IL-7Rα⁺ T cell compartment characteristic of naive T cells, and express large amounts of CD95, IL-2Rβ, CXCR3, and LFA-1.

In addition, in another embodiment, these same T-cells having enhanced reconstitution potential and/or long effective life spans that have been mobilized by administering, for example, G-CSF to a subject in the presence of an E-Selectin inhibitor.

For production of TCR-modified cells, the cells disclosed can be genetically modified using any method known to one of ordinary skill in the art. For example, TCR-carrying constructs can be introduced into the cells via viral transduction (retroviral, lentiviral) or electroporation. Examples of these procedures can be found in the literature and include Bertoletti et al. (2015) T cell receptor-therapy in HBV-related hepatocellularcarcinoma, Oncoimmunology. 2015 Mar. 19; 4(6):e1008354. eCollection 2015, Qasim W et al. (2015) Immunotherapy of HCC metastases with autologous T cell receptor redirected T cells, targeting HBsAg in a liver transplant patient. J Hepatol. 2015 February; 62(2):486-91. doi: 10.1016/j.jhep.2014.10.001. Epub 2014 Oct. 13. and Levine et al (2013) Adoptive transfer of gene-modified T-cells engineered to express high-affinity tcr's for cancer-testis antigens NY-ESO-1 or lage-1, in multiple myeloma (MM) patients post autologous hematopoietic stem cell transplant (ASCT), Cytotherapy, Volume 15, Issue 4, Supplement, Page S13. The differences between TCR-mediated modification and CAR-T cell production have been described in the art in, for example, Qasim et al. (2014) Progress and prospects for engineered T cell therapies. Br J Haematol. 2014 September; 166(6):818-29. doi: 10.1111/bjh.12981. Epub 2014 Jun. 17, and Kershaw et al. (2014) Clinical application of genetically modified T cells in cancer therapy. Clin Transl Immunology. 2014 May 16; 3(5):e16. doi: 10.1038/cti.2014.7. eCollection 2014.

Examples of antigens that can be used as targets in this approach can be found in the CAR-T-related section above. Examples in this category includes the melanocyte differentiation antigens MART-1 and gp100, as well as the MAGE antigens and NY-ESO-1, with expression in a broader range of cancers.

Production of Differentiated T-Cells with T-Cells Having Enhanced Reconstitution Potential and/or a Long Effective Life Span Obtained by Mobilization in the Presence of G-CSF and an E-Selectin Inhibitor.

In one embodiment, the T-cells described herein as having enhanced reconstitution potential and/or long effective life spans, and which have been mobilized by administering G-CSF to a subject in the presence of an E-Selectin inhibitor, can be used for the production of other T-cell populations. Cieri et al., (2013) IL-7 and IL-15 instruct the generation of human memory stem T cells from naive precursors, See comment in PubMed Commons, Blood. 2013 Jan. 24:121(4):573-84. doi: 10.1182/blood-2012-05-431718. Epub 2012 Nov. 15 and comments by Gattinoni et al., (2013) Moving T memory stem cells to the clinic, Blood. 2013 Jan. 24; 121(4):567-8. doi: 10.1182/blood-2012-11-468660; Gattitoni, L. et al. (2011), A human memory T cell subset with stem cell-like properties, Nat Med. 2011 Sep. 18; 17(10):1290-7. doi: 10.1038/nm.2446.

There are different CD8+ T-cell subsets. Naïve, T stem cell (T_{SCM}) and T central memory (T_{CM}) cells circulate and migrate to lymphoid tissue, whereas effector memory T cells (T_{EM}) and effector T cells (T_{EFF}) have the capacity to traffic to peripheral tissues. There are a number of models for the differentiation of CD8+ T cells. Joshi and Kaech, (2008), Effector CD8 T cell development: a balancing act between memory cell potential and terminal differentiation. J. Immunol. 180, 1309-1315. One model is the linear model for differentiation of CD8+ T cells, which proposes that, following activation of a naïve T cell, there is a progressive differentiation through three major circulating subsets of T cells (T_{SCM}, T_{CM} and T_{EM}), with T_{EFF} representing the terminally differentiated T cells. Targeting different T-cell subsets could increase efficacy and persistence of genetically modified T-cell therapies. Accordingly, non-limiting examples of cells that can be derived from the cells disclosed herein include T_{CM}, T_{EM}, T_{EFF}, Tregs. Tregs are immune suppressive cells that can act to dampen down T cell responses. Certain human autoimmune, infectious, and allergic diseases are associated with impaired regulatory T-cell function. Adoptive transfer of Tregs is considered a possible treatment for these disorders. For examples of references addressing the uses for Tregs see, e.g., Bluestone et al. (2015) The therapeutic potential of regulatory T cells for the treatment of autoimmune disease, Expert Opin Ther Targets. 2015 Apr. 16:1-13 and Taams et al. (2006) Regulatory T cells in human disease and their potential for therapeutic manipulation, Immunology. 2006 May; 118(1): 1-9. For example, since colitis is caused by a patient's own T cells reacting against protein targets like CEA (carcinoembryonic antigen) in the colon, Tregs can suppress these immune responses and restore a healthy colon. During immune homeostasis, Tregs counterbalance the actions of autoreactive T_{EFF} cells, thereby participating in peripheral tolerance. This process is disregulated in a number of diseases, including Type 1 diabetes mellitus, where there is an imbalance in the number or function of Tregs vs. T_{EFFS}.

### Compositions

Within the scope of the disclosure are also compositions comprising the T-cells that are mobilized by G-CSF (or another mobilizer) in the presence of at least one E-Selectin inhibitor. In one embodiment, these compositions comprise the disclosed T-cells and at least one antibody. In one embodiment, the at least one antibody is chosen from antibodies against CD44, CD62L, CD45RO, CCR7, CD45RA, CD62L, CD27, CD28, IL-7Rα, CD95, IL-2Rβ, CXCR3, and LFA-1. In one embodiment, the compositions comprise the disclosed T-cells in an artificial cell growth medium. In one embodiment, this medium permits the cell's growth, In one embodiment, this medium permits cell differentiation. In one embodiment, the at least one E-Selectin inhibitor is GMI-1271 and the at least one mobilizer is G-CSF. In one embodiment, mobilization is done by blocking CXCR4 in combination with VLA-4. In one embodiment of these compositions, the peripheral blood naïve/memory stem cells (i.e. (T_{N}/T_{CM}/T_{SCM}) T-cells with enhanced reconstitution potential and/or longer life spans are CD62L^{high}CCR7+ cells that have been mobilized by G-CSF (or another mobilizer) in the presence of at least one E-Selectin inhibitor. In some embodiments, the T-cells of interest are CD8+CD62L^{high} T-cells that have been mobilized by G-CSF (or another mobilizer) in the presence of at least one E-Selectin inhibitor. In some embodiments, the T-cells of interest are CD8⁺CD62L^{high}CCR7+ T-cells. In one embodiment, the T-cells of interest can be found within a CD45RO−, CCR7+, CD45RA+, CD62L+, CD27+, CD28+ and IL-7Rα+ T cell compartment characteristic of naive T cells, express large amounts of CD95, IL-2Rβ, CXCR3, and LFA-1, and have been mobilized by G-CSF (or another mobilizer) in the presence of at least one E-Selectin inhibitor.

In one embodiment, the disclosure provides a pharmaceutical composition comprising an anti-tumor effective amount of a population of modified autologous human T cells, wherein the T cells comprise a nucleic acid sequence that encodes a chimeric antigen receptor (CAR), and wherein the T-cells from which the CAR-T cells are engineered are produced according to the methods disclosed herein. In one embodiment, the T-cells from which the CAR-T cells were made are CD8⁺CD62L^{high} T-cells. In one embodiment, the T-cells from which the CAR-T cells were made are CD62Lhigh/+CCR7+. In some embodiments, the T-cells of interest are CD8⁺CD62L^{high}CCR7+ T-cells. In another embodiment, the T-cells of interest are can be found within a CD45RO−, CCR7+, CD45RA+, CD62L+, CD27+, CD28+ and IL-7Rα+ T cell compartment characteristic of naive T cells, express large amounts of CD95, IL-2Rβ, CXCR3. and LFA-1, and have been mobilized by G-CSF (or another mobilizer) in the presence of at least one E-Selectin inhibitor.

In one embodiment, the anti-tumor effective amount of T-cells is 10⁴ to 10⁹ cells per kg body weight of a human in need of such cells. In one embodiment, the anti-tumor effective amount of T cells is 10⁷ to 10⁸ cells per kg body weight of a human in need of such cells. In one embodiment, the anti-tumor effective amount of T cells is 10⁵ to 10⁶ cells per kg body weight of a human in need of such cells.

### Examples

### I. Increased Mobilization of CD8+CD62Lhi CD44+ Cells

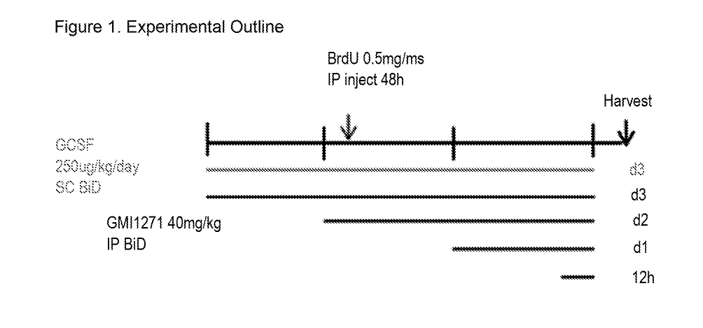

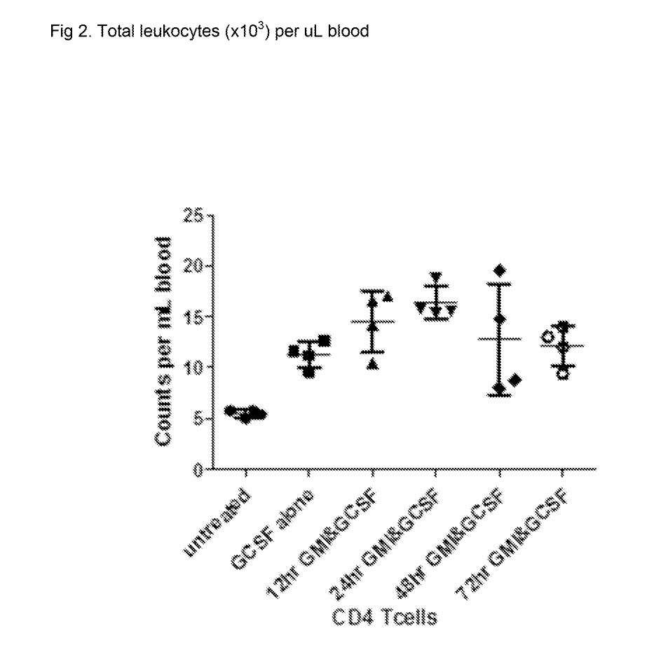

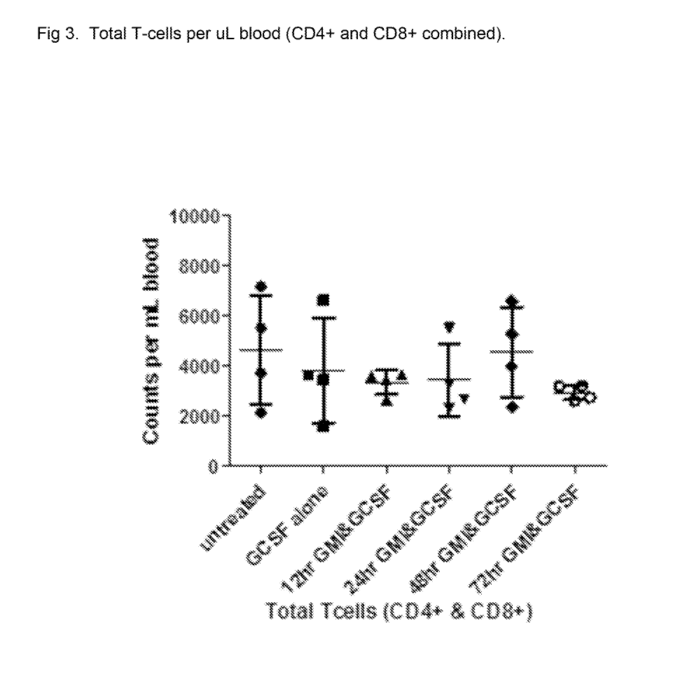

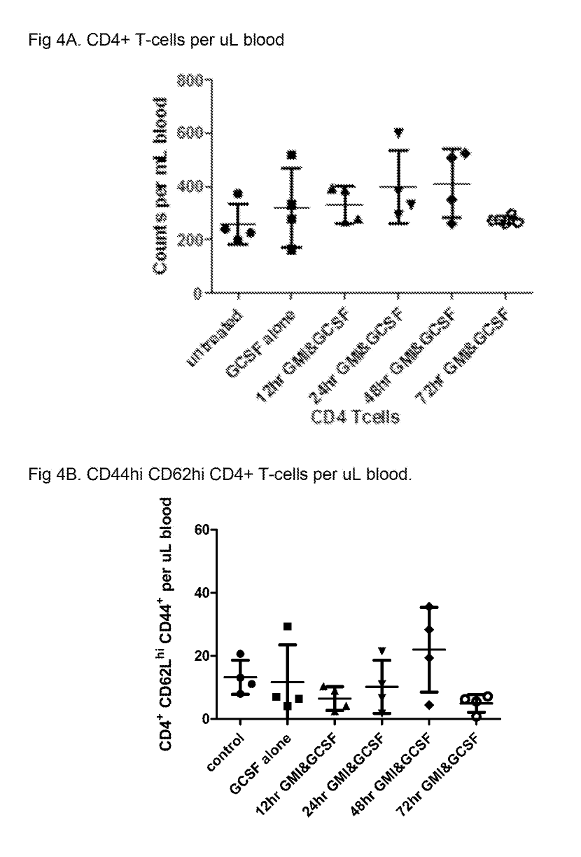

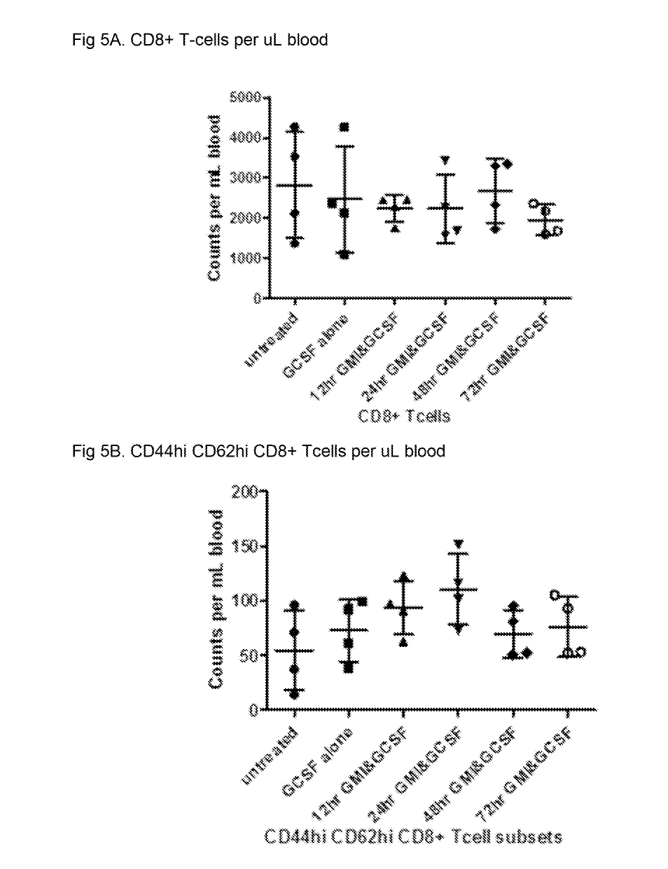

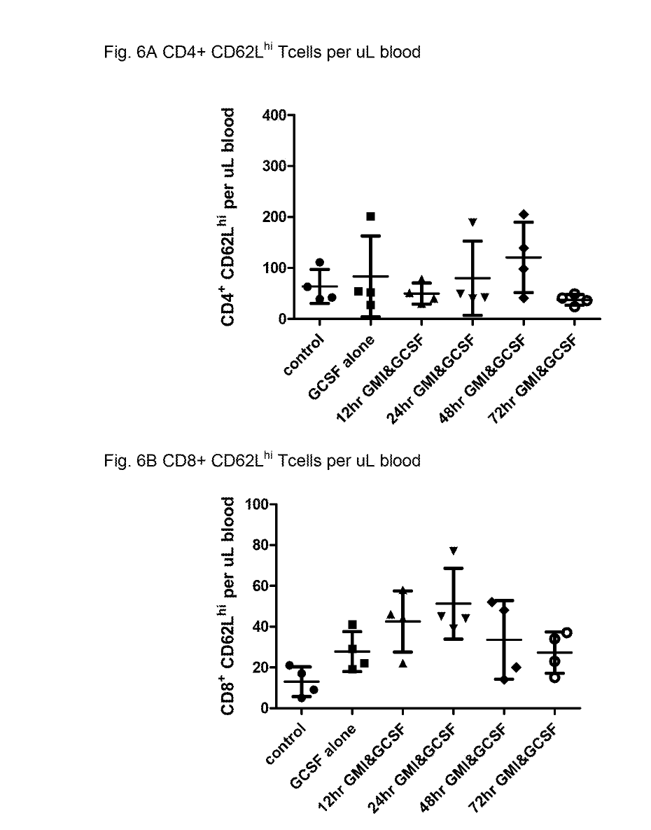

The following classification was used in the following experiments. Of all the CD8+ cells the Tₙₐᵢᵥₑ, population is CD62hi CD44′; the T_{CM/SCM} population is CD62hi CD44+; and the TEM population is CD44+CD62⁻ population. An increase in the TCM/SCM mouse cell population is observed below (see FIGS. 1 through 6).

Cohorts of mice (C57bl/6 adult 10 week old males) were administered G-CSF (Filgastim, Amgen) at 125ug/kg/injection bidaily subcutaneous injections for a total of 72 hours before sacrifice (with non-mobilised control group #1 receiving saline injection instead).

The E-selectin mimetic antagonist GMI-1271 (bidaily intraperitoneal injections at 40 mg/kg/injection), was co-administered to these mice at specified timepoints prior to sacrifice with a final GMI-1271 injection always occurring exactly 1 hour prior to sacrifice. Generally all mice received injections at 9 am and 6 pm every day with n=4 per group. The injection groups are outlined in FIG. 1 and are detailed below.

The mouse groups were: Group 1. non-mobilised control, Group 2. G-CSF alone control, Group 3. 14 hours GMI-1271, Group 4. 24 hours GMI-1271, Group 5. 48 hours GMI-1271, Group 6. 72 hours GMI-1271

After 3 days (72 hours) G-CSF administration mice were euthanised administration and heparinised blood collected by cardiac puncture. Total blood leukocytes counts were enumerated using an automated haematological cell counter (Beckman-Coulter KX-21), For flow cytometry, red cells were first lysed using ammonium lysis then blood leukocytes washed in phosphate buffered saline (PBS) containing 2% fetal calf serum and incubated on ice in the presence of excess FcBlock (CD16/CD32 hybridoma 2.4G2 supernatant to block endogenous Fc receptors) following the methodology in Winkler et al., Nature Medicine 2012. Cells were stained using the antibodies CD4-pacific blue, CD8-peCY7, CD62L-BV605, CD44-AF700 at a final concentration of approx 5 million leukocytes per 100 uL volume containing 0.5 ug of each of the above conjugated monoclonal antibodies. All conjugated antibodies were purchased from Biolegend, CA. After 40 minutes incubation on ice, cells were washed to remove excess antibody (by addition of 1 mL PBS, centrifugation at 370×g for 5 minutes at 4 C, removal of supernatant and resuspension of pellet in 100 uL PBS with 2% fetal calf serum). Flow cytometry of cells was performed on the 8 colour CYAN (Beckman Coulter) using unstained blood and single colour controls to set voltages and gating strategy.

CD8+ Tcell subsets were divided based on CD62 and CD44 staining. With CD8+ naive T cells being CD62hi CD44−, CD8+T CM/SCM cells being CD62hi CD44+ double positive and TEM being CD44+ and CD62 negative following routine methodology.

Data was later analysed using the Flow Jo software and collated in excel software. Figures and statistics were by ANOVA using prism software.

### II. Increased Mobilization of CCR7 Cells

Methods and Materials:

1) Test Animals:
1. Species: Mouse
2. Strain: Balb/C and C57BV6
3. Age and gender 7-9 weeks, Female
4. Source: Harlan Laboratories
5. Total number: 25

2) Test Articles:

### Preparation of Test Articles:

G-CSF and GM-1271 in powder form. The test agents were reconstituted with saline 600 μg of G-CSF was dissolved in 6.5 mL of sterile saline giving a concentration of 92.307 μg/mL. Animals were administered with 0.1 mL of dose solution per time point giving the dose concentration for G-CSF was 9.23 μg per mouse per time point.

70 mg of GM-1271 was reconstituted with 7 mL of sterile saline giving a concentration of 10 mg/mL. Animals were administered with 0.1 mL of dose solution per time point giving the dose concentration for GM-1271 was 1 mg per mouse per time point.

### 3) Animal Treatment:

1. Treatment of animals was in accordance with the study protocol and also in accordance with Noble SOPs which adhere to the regulations outlined in the USDA Animal Welfare Act (9 CFR Parts 1, 2 and 3) and the conditions specified in the Guide for the Care and Use of Laboratory Animals (ILAR publication, NRC, 2011, The National Academies Press). The Noble Institutional Animal Care and Use Committee (IACUC) approved the study protocol prior to finalization to insure compliance with acceptable standard animal welfare and humane care.

TABLE 1
Experimental Design: Dosing and Sample Collection Schedule

| | | | Dose | Route and | Day | Blood collection and |
|---|---|---|---|---|---|---|
| Group # | N | Treatment | Concentration | Volume | Schedule | serum preparation |
| 1 | 5 | No Treatment | | IP/0.1 mL | qdX3 | Collect whole blood |
| 2 | 5 | Saline | | IP/0.1 mL | qdX3 | in EDTA tubes. Red |
| 3 | 5 | G-CSF | 10 μg | IP/0.1 mL | qdX3 | blood cells will be |
| 4 | 5 | GI 1271 | 1 mg | IP/0.1 mL | qdX3 | lysed and counted for |
| 5 | 5 | G-CSF + | 10 μg/1 mg | IP/0.1 mL | qdX3 | leukocytes |
| | | GMI-1271 | | | | |

5) Experimental Procedures:

### 1. Preparation of Peripheral Blood Mononuclear Cells (PBMC):

To purify PBMCs, red blood cells (RBCs) in the whole blood were lysed using ACK Lysing Buffer (Life Technologies, A1049201). Whole blood was mixed with ACK Lysing Buffer at a 1: 25 ratio and incubated at room temperature on shaker for 5 min. The cells were centrifuged at 350 g for 7 min and the supernatant was discarded. The purified PBMCs were suspended in PBS containing 2% FBS. Cell counts were performed, pelleted at 250×g for 10 minutes and suspended in 5 ml HBSS containing containing Ca2+ and Mg2+(Life Technologies catalog #14025-092) plus 0.05% bovine serum albumin (BSA) to 2-3×10^{e} cells/mL for antibody staining.

### 2. Antibody Staining

Add 100 ul cells (2-3×105 cells) per tube (5 mL polypropylene round-bottom tubes (BD Falcon tubes #352063, VWR catalog #60819-728). Add indicated amounts of Alexa Fluor 488® labeled antibodies as indicated in Table 3 and immediately place on ice.
Place tubes at 4° C. for 1 hour then add 2 mL cold HBSS/BSA to each tube and pellet cells 250×g for 10 minutes. Pour off supernatant and suspend the cell pellet in 1 mL cold HBSS/BSA. Pellet cells 250×g for 10 minutes, pour off supernatant, and suspend cell pellet in 250 microliters HBSS/BSA. Add 250 microliters 2% formaldehyde (Polysciences, Inc. catalog #04018, 10% ultrapure, EM grade, methanol-free). Proceed to flow cytometry.

### 3. Flow Cytometry

Fluorescence was acquired via blue laser excitation at 488 nm with emission at 530 nm using preset lasers equipped on the Attune@ NxT Accoustic Focusing Cytometer (Life Technologies). 2×104 events were collected for each sample and analyzed with the use of the Attune® NxT Cytometric Software v2.1. Results were recorded as median fluorescent intensity, % cells positive for a given marker, and as cell concentration of expressing the specific marker per mL.

TABLE 3
Antibody staining guide

| Antibody | Source | Amount |
|---|---|---|
| Anti-mouse CCR7 clone 4B12 | Biolegend 120110 | 1 microgram |
| Anti-mouse CD62L clone | Biolegend 104420 | 0.25 microgram |
| MEL14 | | |
| Anti-mouse/human CD44 clone | Biolgend 103016 | 1 microgram |
| IM7 | | |
| Anti-mouse CD4 clone GK1.5 | Biolegend 100423 | 0.05 microgram |
| Anti-mouse CD8 clone 53-6.7 | Biolegend 100723 | 0.25 microgram |
| IgG2b κ isotype control clone | Biolegend 400625 | 0.25 microgram |
| RTK4530 | | |
| IgG2a κ isotype control clone | Biolegend 400525 | 0.25 microgram |
| RTK2758 | | |

These experiments were done in both Balb/c (top table) and BL6 (bottom table) mouse strains. The results show that, in both strains, the addition of GMI-1271 to G-CSF increases the mobilization of CCR7+ cells.

| | Total | | | | | |
|---|---|---|---|---|---|---|
| | cells/mL | Cells/mL(10⁵) | | | | |
| Group | (10⁶ + SD) | CD4 | CD8 | CD44 | CD62L | CCR7 |
| Naïve | 1.42 ± 0.34 | 2.20 | 0.67 | 6.76 | 1.22 | 1.99 |
| Saline | 1.51 ± 0.13 | 3.00 | 0.98 | 7.64 | 1.45 | 2.76 |
| G-CSF | 2.67 ± 0.64 | 3.43 | 1.32 | 14.88 | 1.59 | 4.00 |
| GMI-1271 | 1.01 ± 0.46 | 1.56 | 0.49 | 4.76 | 0.74 | 1.68 |
| G-CSF + | 4.31 ± 2.15 | 7.07 | 2.35 | 21.76 | 2.33 | 6.80 |
| GMI-1271 | | | | | | |
| Naïve | 0.97 ± 0.10 | 0.37 | 1.08 | 1.67 | 0.10 | 0.10 |
| Saline | 1.47 ± 0.59 | 0.46 | 1.91 | 3.14 | 0.21 | 0.21 |
| G-CSF | 5.98 ± 2.25 | 1.10 | 10.00 | 12.65 | 0.09 | 0.60 |
| GMI-1271 | 1.89 ± 0.40 | 0.43 | 1.74 | 3.56 | 0.19 | 0.23 |
| G-CSF + | 5.06 ± 0.78 | 1.07 | 3.73 | 8.18 | 0.10 | 2.78 |
| GMI-1271 | | | | | | |

We claim:

1. A method of mobilizing to the peripheral blood from a subject T-cells that are either Tₙₐᵢᵥₑ T_{CM} T_{SCM}, CD62L^{high}CCR7⁺, CD8⁺CD62L^{high} CCR7⁺, CD8⁺CD62L^{high}, CD44⁻CD8⁺CD62L^{high}, or CD44⁺CD8⁺CD62L^{high}, and combinations thereof, with enhanced reconstitution potential and/or a long life span, the method comprising administering to the subject at least one mobilizer in combination with at least one E-selectin inhibitor.

2. The method of claim 1, wherein the at least one mobilizer is G-CSF.

3. The method according to any one of claims 1 and 2, wherein the at least one E-selectin inhibitor is GMI-1271.

4. The method according to any one of claims 1, 2, and 3, wherein the G-CSF is administered at a dose from 0.5 μg/kg/day to 50 μg/kg/day.

5. The method according to any one of claims 1 through 4, wherein the T-cells are CD62L^{high}CCR7⁺ cells.

6. A method of modulating an immune response in a subject in need thereof, wherein the subject suffers from at least one condition selected from cancers, infectious diseases, autoimmune diseases, GVHDs, and transplantations, the method comprising administering to the subject T-cells that are either Tₙₐᵢᵥₑ, T_{CM} T_{SCM}, CD62L^{high}CCR7⁺, CD8⁺CD62L^{high}, CD8⁺CD62L^{high}CCR7⁺, CD44⁻CD8⁺CD62L^{high}, or CD44⁺CD8⁺CD62L^{high}, and combinations thereof, with enhanced reconstitution potential and/or a long life span.

7. The method of claim 6, wherein the at least one mobilizer is G-CSF.

8. The method according to any one of claims 6 and 7, wherein the at least one E-selectin inhibitor is GMI-1271.

9. The method according to any one of claims 6, 7, and 8, wherein the G-CSF is administered at a dose from 0.5 μg/kg/day to 50 μg/kg/day.

10. The method according to any one of claims 6 through 9, wherein the T-cells are CD62L^{high}CCR7⁺ cells.

11. A method of producing CAR-T cells with enhanced reconstitution potential and/or a long life span, wherein the CAR-T cells are produced according to any one of claims 1 through 5.

12. The method of claim 11, wherein the at least one mobilizer is G-CSF.

13. The method according to any one of claims 11 and 12, wherein the at least one E-selectin inhibitor is GMI-1271.

14. The method according to any one of claims 11, 12, and 13, wherein the G-CSF is administered at a dose from 0.5 μg/kg/day to 50 μg/kg/day.

15. The method according to any one of claims 11 through 14, wherein the T-cells are CD62L^{high}CCR7⁺ cells.

16. A method of producing TCR-modified cells with enhanced reconstitution potential and/or a long life span, wherein the TCR-modified cells are produced according to any one of claims 1 through 5.

17. The method of claim 16, wherein the at least one mobilizer is G-CSF.

18. The method according to any one of claims 16 and 17, wherein the at least one E-selectin inhibitor is GMI-1271.

19. The method according to any one of claims 16, 17, and 18, wherein the G-CSF is administered at a dose from 0.5 μg/kg/day to 50 μg/kg/day.

20. The method according to any one of claims 16 through 19, wherein the T-cells are CD62L^{high}CCR7⁺ cells.

21. A method of treating cancer, infections, autoimmune diseases in a subject in need thereof, the method comprising administering to the subject cells are produced according to any one of claims 1 through 5 and 11 through 20.

22. The method of claim 21, wherein the at least one mobilizer is G-CSF.

23. The method according to any one of claims 21 and 22, wherein the at least one E-selectin inhibitor is GMI-1271.

24. The method according to any one of claims 21, 22, and 23, wherein the G-CSF is administered at a dose from 0.5 μg/kg/day to 50 μg/kg/day.

25. The method according to any one of claims 21 through 24, wherein the T-cells are CD62L^{high}CCR7⁺ cells.

26. A method of producing differentiated T-cells, the method comprising culturing cells that are produced according to any one of claims 1 through to 5 and 11 through 20.

27. A composition comprising a population of T-cells, wherein the T-cells have been produced according to any one of claims 1 through 5 and 11 through 20.

28. The composition of claim 27, further comprising at least one antibody chosen from antibodies against CD44, CD62L, CD45RO, CCR7, CD45RA, CD62L, CD27, CD28, IL-7Rα, CD95, IL-2Rβ, CXCR3, and LFA-1.

29. The composition according to any one of claims 27 and 28, further comprising artificial cell growth medium.
